(19) United States
(12) Patent Application Publication
(10) Pub. No.: US 2026/0020776 A1
(43) Pub. Date: Jan. 22, 2026

(54) METHOD AND SYSTEM FOR ANALYSIS OF BREATH RATE VARIATIONS

(71) Applicant: Calibre Biometrics Inc., Wellesley Hills, MA (US)

(72) Inventor: Udi E. Meirav, Waban, MA (US)

(73) Assignee: Calibre Biometrics Inc., Wellesley Hills, MA (US)

(21) Appl. No.: 19/272,489

(22) Filed: Jul. 17, 2025

Related U.S. Application Data
(60) Provisional application No. 63/672,836, filed on Jul. 18, 2024.

(51) Int. Cl. A61B 5/097; A61B 5/00; A61B 5/08; A61B 5/087; A61B 5/091

(52) U.S. Cl. CPC A61B 5/097; A61B 5/0816; A61B 5/087; A61B 5/091; A61B 5/4818; A61B 5/746

(57) ABSTRACT

Techniques for gathering and analyzing respiratory information concerning a subject are disclosed. The techniques may involve determining a plurality of metrics concerning respiration by a subject over time. Metrics concerning respiration by the subject may be compared to determine one or more respiratory features of the subject. Such respiratory feature(s) may then be used to identify at least one respiratory condition which the subject exhibits or is at risk of exhibiting.

### CROSS REFERENCE TO RELATED APPLICATIONS

This application claims the benefit of priority under 35 U.S.C. § 119 (e) of U.S. Provisional Patent Application Ser. No. 63/672,836, filed Jul. 18, 2024, entitled “METHOD AND SYSTEM FOR ANALYSIS OF BREATH RATE VARIATIONS”, the entirety of which is incorporated herein by reference.

### TECHNICAL FIELD

Embodiments of the present disclosure generally relate to collecting and analyzing respiratory data.

### BACKGROUND

The duration of each breath in normal human beings varies significantly. A typical breath duration for a healthy adult at rest is generally 4-5 seconds, corresponding to a breath rate of 12-15 breaths per minute (BPM). However, under exertion or stress, breath duration becomes much shorter and can be less than 1 second, namely a rate of over 60 BPM. On the other hand, breathing can be controlled to achieve much slower rates, especially during, or as a result of, deliberate practice. There are various training disciplines that teach slow breathing and breath holding, resulting in significantly lower breath rates, as low as a 2-3 BPM and at times even less than 1 BPM. Some trained individuals are able to hold their breath for well over 5 minutes. Thus, human breath rate varies over a dynamic range of more than an order of magnitude.

### SUMMARY

Some embodiments provide for a system for gathering respiratory information concerning a subject, the system comprising: a mask, comprising a shell and a seal, the seal being arranged to contact a face of the subject and to circumscribe an area of the face when the mask is worn by the subject, the area including a mouth of the subject, the seal and the shell separating an interior space about the area of the face from an ambient space, the shell having a plurality of breathing apertures configured to allow air to flow between the ambient space and the interior space; at least one differential pressure sensor, coupled to the mask, configured to measure a difference in air pressure between the ambient space and the interior space; at least one computer hardware processor communicatively coupled to the at least one differential pressure sensor; and at least one non-transitory computer readable storage medium storing instructions which, when executed by the at least one computer hardware processor, cause the at least one computer hardware processor to: determine, based at least in part on measurements by the at least one differential pressure sensor, a plurality of metrics each concerning one or more breaths taken by the subject; compare the plurality of metrics to determine one or more respiratory features of the subject; and based at least in part on the one or more respiratory features, identify at least one respiratory condition which the subject exhibits or is at risk of exhibiting.

### BRIEF DESCRIPTION OF THE DRAWINGS

Various aspects and embodiments of the invention are described herein with reference to the following figures. It should be appreciated that these are schematic figures not necessarily drawn to scale, but rather intended to explain the key features and operating principles of the invention. In the figures:

FIG. 1A is an example system for recording and analyzing breathing data of a subject, according to some embodiments;

FIG. 1B is a block diagram depicting components of an example breathing data analysis system, according to some embodiments;

FIG. 2 is a diagram of an example mask which may be worn by a subject during breathing, according to some embodiments;

FIG. 3A is a diagram depicting an alternative example mask worn by a subject during breathing, according to some embodiments;

FIG. 3B is a view of the example mask of FIG. 3A, detached from the subject, according to some embodiments;

FIG. 4A depicts example air flow data concerning a subject, according to some embodiments;

FIG. 4B depicts example air flow data concerning subject that is exercising, according to some embodiments;

FIG. 5 is a scatterplot of example breath duration data concerning a subject, according to some embodiments;

FIG. 6 is a frequency analysis of breathing data concerning a subject, according to some embodiments;

FIG. 7 is a flow chart of an example process that may be performed to record and analyze breathing data of a subject, according to some embodiments; and

FIG. 8 illustrates a block diagram of an illustrative computing system that may be used in implementing some embodiments.

### DETAILED DESCRIPTION OF THE INVENTION

The Assignee has appreciated that breath is a cyclical process and under most conditions the duration and volume of consecutive breaths are similar. However, the Assignee has also appreciated that under certain conditions (e.g., disease states, stress, exertion, exercise, etc.) breaths may exhibit changes in respiratory metrics, and that variability of respiratory metrics is a valuable biometric indicator of health and wellness, and may be used in the diagnosis and monitoring of diseases and respiratory function.

Respiratory metrics may additionally vary within a single breathing cycle, for example, the modulation of flow rate within each breath cycle is generally not a simple sinusoidal function of time, rather, more typically, showing a rapid rise at the beginning of each tidal cycle, followed by a longer slowdown. The specific temporal profile of each breath may vary significantly, between individuals and even for the same person. The Assignee has further appreciated that such variations too may be analyzed and used in the diagnosis and monitoring of diseases and respiratory functions.

The Assignee has also appreciated that, despite the significance of these temporal characteristics of breath as a diagnostic tool and as a measure of wellness, they are seldom collected and analyzed, especially outside of intensive care facilities. This is likely due to the current equipment, time and settings required for collecting such data. Existing equipment for collecting breath data commonly requires clinical settings and/or the user to be attached to monitoring equipment such as through wires or tubes. Thus, breath data is typically collected over short time periods and is not collected during everyday life or activities that are more representative of an individual's ongoing health and condition.

The Assignee has further appreciated that self-contained, wearable breath monitoring systems may be used to collect breathing data of subjects over extended periods of time and outside of clinical settings. Accordingly, the Assignee has developed techniques for monitoring the breathing of a subject and employing respiratory metrics to identify one or more conditions which the subject exhibits or may be about to exhibit. In this respect, the techniques disclosed herein may employ a system for measuring the breathing of a subject to collect breathing data, and analyzing the collected breathing data to determine one or more respiratory metrics and/or features concerning the subject. The respiratory metrics and/or features may be analyzed to identify one or more conditions that the subject exhibits or may exhibit in the future.

In some embodiments, breathing data of a subject may be collected using a mask equipped with one or more differential pressure sensors. The mask may be lightweight and untethered from additional equipment, allowing it to be worn continuously for extended periods and during everyday activities (e.g., sleep, exercise, etc.). The mask may fit over the face of the subject and at least partially cover the mouth and/or nose of the subject. The mask may include an interior space that is separated from the ambient environment. The mask may include one or more apertures that allow for airflow between the interior space and the ambient environment. The differential pressure sensor may measure the difference in air pressure between the ambient environment and the interior space of the mask, representing the breathing of the subject. Examples of masks which may be used in conjunction with the techniques disclosed herein are described in U.S. Pat. No. 11,375,950, which is incorporated by reference herein in its entirety.

In some embodiments, breathing data recorded using a mask and/or other breathing apparatus is analyzed by a breathing data analysis system. In some embodiments, the breathing data analysis system may be integrated with the mask, while in some embodiments, the breathing data analysis system may be separate from the mask. In some embodiments, the breathing data provided to the breathing data analysis system relates to differential pressure data recorded while the subject wears the mask, while in some embodiments, the breathing data provided to the breathing data analysis system relates to air flow data determined based at least in part on differential pressure data.

In some embodiments, respiratory metrics for a subject may be determined based on recorded breathing data. For example, a breath rate, air flow, breath duration, inhaled volume, exhaled volume, peak inhalation flow rate, peak exhalation flow rate and/or inspiratory to expiratory ratio (I/E ratio) may be determined based on recorded breathing data of a subject.

The Assignee has appreciated a correlation between the value and significance of respiratory metrics in identifying conditions which are or may be exhibited by subjects and the amount of breathing data used to determine such metrics. That is, the Assignee has appreciated that, in general, the more the amount of breathing data recorded concerning a subject, the more meaningful and valuable the respiratory metrics determined based on that breathing data are in identifying conditions which are or may be exhibited by the subject, and the more insights concerning the subject's health and wellness that may be gleaned. As such, some embodiments provide for breathing data concerning a subject to be recorded over an extended period, such as by using a self-contained mask which may be worn continuously during sleep, exercise, or otherwise during everyday life, so that breathing data may be recorded over an extended period, and such metrics and insights may be captured. Such data, recorded over extended periods during everyday life, may provide insights which could not otherwise be obtained in clinical settings where the breathing of the subject may be monitored for a short period. Additionally, because the data is recorded continuously, during everyday life, the subject may be more likely to exhibit natural breathing and less likely to subconsciously change their breathing patterns, as is common in clinical settings. This natural breathing allows for the identification and analysis of respiratory conditions exhibited by a subject.

Of course, it should also be appreciated that breathing data concerning a subject need not be recorded over an extended period using a self-contained mask. Any of numerous approaches, techniques, devices and/or systems may be used to record breathing data, over any suitable duration(s), as the invention is not limited in this respect.

In some embodiments, breathing data may be analyzed to determine respiratory metrics over various periods. For example, respiratory metrics may be determined over a predetermined time period (e.g., 1 second, 5 seconds, 10 seconds, 30 seconds, 1 minute, 5 minutes, 10 minutes, 30 minutes, 1 hour, 3 hours, 5 hours, 10 hours, greater than 10 hours, and/or any time period between 1 second and 10 hours). Each time period may correspond to a plurality of breaths or breathing cycles. Additionally or alternatively, respiratory metrics may be determined over a predetermined number of breath cycles (inhalation and exhalation) of a subject (e.g., 1 breath cycle, 2 breath cycles, 3 breath cycles, 5 breath cycles, 10 breath cycles, 20 breath cycles, 30 breath cycles, 50 breath cycles, 100 breath cycles, 500 breath cycles, 1,000 breath cycles, 5,000 breath cycles greater than 5,000 breath cycles, and/or between 1 breath cycle and 5,000 breath cycles). Breath metrics may additionally or alternatively be determined for portions of a breath cycle (e.g., inhalation and/or exhalation).

In some embodiments, respiratory metrics concerning a subject may be analyzed to determine one or more respiratory features for a subject. Such features may include statistical features such as a mean, median, variance, standard deviation, percentiles, maximum value, and/or minimum value of a respiratory metric over a period (e.g., time period or number of breath cycles). In some embodiments, the respiratory features determined for a subject may include one or more of a skewness and/or kurtosis for a respiratory metric over a period (e.g., time period or number of breath cycles).

In some embodiments, respiratory metrics concerning different breaths over time may be compared to determine one or more respiratory features for a subject. As one example, respiratory metrics determined from breathing data recorded during different time periods may be compared. Respiratory metrics determined from breathing data recorded at consecutive periods may be compared (e.g., successive breathing cycles of a subject, consecutive periods of time, etc.) Alternatively or additionally, respiratory metrics determined from breathing data recorded from non-consecutive periods may be compared. Of course, embodiments of the invention need not identify respiratory features by comparing respiratory metrics determined from breathing data recorded in different time periods. For example, in some embodiments, respiratory features may be identified by comparing respiratory metrics determined from breathing data recorded in a single breathing cycle. Respiratory features concerning a subject may be identified based on respiratory metrics determined from breathing data recorded during any suitable number of time periods, as the invention is not limited in this respect.

In some embodiments, respiratory features may be determined from the comparison of respiratory metrics and/or other respiratory features, and such features may include one or more respiratory variabilities for the subject. These respiratory variabilities may include a breath rate variability, a breath duration variability, an inhaled volume variability, an exhaled volume variability, a peak inhalation flow rate variability, a peak exhalation flow rate variability and/or an I/E ratio variability, among other variabilities that may be determined based on respiratory metrics. The respiratory variabilities may represent how much a particular respiratory metric changes between different periods (e.g., time periods and/or breath cycles). As described in further detail below, the Assignee has appreciated that irregularities and non-uniform breathing may be indicative of various respiratory conditions.

In some embodiments, respiratory variabilities may be determined based on comparisons of respiratory metrics and/or other respiratory features for a given period. For example, respiratory variabilities may be a standard deviation of a respiratory metric over a given period, a ratio of the standard deviation of a respiratory metric over a given period to the mean of the respiratory metric, a normalized score representing the uniformity of a respiratory metric over a given period (e.g., 100 representing perfect uniformity and lower scores representing less uniform breathing), and/or a distribution of the difference of respiratory metrics over successive periods (e.g., time periods, or breath cycles) determined using Poincaré analysis.

In some embodiments, respiratory features may include one or more features determined based on a frequency, harmonic, and/or Fourier analysis of breathing data and/or respiratory metrics. For example, by analyzing breathing data in the frequency domain, the regularity of breathing can be determined, as narrow peaks at different frequencies represent highly regular breathing, while broad peaks across many frequencies represent irregular breathing.

The Assignee has appreciated that by determining respiratory metrics and/or respiratory features from breathing data collected over extended periods, respiratory conditions may be determined for a subject. The Assignee has appreciated that many respiratory conditions are correlated with irregular breathing patterns such as changes in breathing between breaths and/or over time, and such conditions may be identified through respiratory metrics and/or features determined from breathing data. Such conditions may include one or more of: Obstructive Sleep Apnea (OSA), Breath Pattern Disorder (BPD), and/or Cheyne-Stokes respiration (CSR). In some embodiments, non-medical conditions may be identified and/or analyzed based on respiratory metrics and/or features, such as exercise, respiratory exertion and/or stress. It may be determined that a subject has or may exhibit in the future a particular condition when one or more respiratory features satisfy one or more criteria associated with the particular condition. Alternatively, it may be determined that a subject has or may exhibit in the future a particular condition when a combination of respiratory features satisfy one or more criteria associated with the particular condition (e.g., when a predetermined number of respiratory features exceed a threshold; when feature X is lower than a threshold while one or more of features A, B, and C exhibit a predetermined relationship, etc.).

It should be appreciated that, as used herein, the term “condition” does not necessarily refer to a state which is dichotomous, in that it is one that a subject either has, exhibits or manifests or does not (or is likely to have, exhibit or manifest or is not). For example, the term “condition” as used herein may refer to a physiological state residing along a continuum in a way that is quantifiably measurable, such as using degrees, gradients, ranks, percentages, scores, etc. Such a condition may, for example, refer to an extent to which a subject is undergoing physical exertion (e.g., during a workout), an extent to which a subject is observed to progress toward a particular status, and/or an extent to which a subject has, exhibits or manifests (or does not have, exhibit or manifest) any other suitable characteristic(s) and/or quality(ies). As another example, a “condition” may refer to a state which is one of more than two possible alternatives. As another example, a “condition” may refer to a likelihood of a subject satisfying or not satisfying one or more criteria at some point in the future. As yet another example, a “condition” may refer to a combination of states, each reflecting any of numerous factors, alternatives and/or possibilities. It should be appreciated, then, that the term “condition” is intended to be interpreted broadly, to encompass any state(s) concerning or relating to a subject, whether recognized now or in the future.

In some embodiments, the techniques described herein may be used to determine whether a subject has or is likely to have Obstructive Sleep Apnea (OSA). OSA is a major health concern that affects a significant fraction of the adult population. Patients suffering from OSA experience a blocked respiratory tract occurring multiple times during sleep, typically due to relaxed soft tissue collapsing during inhalation. This results in highly irregular breathing, and in particular repeated respiratory stoppage (apnea), which frequently interrupts their sleep and is associated with a range of deleterious effects on a patient's overall health over time. OSA presents itself in particular non-uniformities of breath rate and volume during sleep, and its severity is directly and quantitatively linked to these statistical signatures. In some embodiments, the techniques described herein may be used to determine whether a patient has or is likely to have OSA. Using the techniques described herein, OSA may be detected and precisely quantified during sleep, without requiring the subject to be tethered to stationary equipment or to spend a night in a specialized laboratory. For example, a subject may wear a device for collecting breath data (e.g., a mask equipped with a differential pressure sensor or other suitable device) during sleep to record breathing data for the subject. The breathing data for the subject may be analyzed to determine respiratory metrics for the subject (e.g., a breath rate, an inhaled volume, and/or an exhaled volume), and respiratory metrics determined at different periods (e.g., different breaths, different times, etc.) may be compared to determine respiratory features for the subject. It may be determined that the subject is experiencing OSA or is likely to be experiencing OSA when one or more respiratory features for the subject satisfy one or more criteria. For example, it may be determined that the subject is or is likely to be experiencing OSA when a breath rate variability, an inhaled volume variability, and/or an exhaled volume variability exceeds respective thresholds. Additionally, or alternatively, it may be determined that the subject is experiencing or may be likely to experience OSA when a combination of respiratory features for the subject satisfy one or more criteria, such as if two or more of a breath rate variability, an inhaled volume variability, and/or an exhaled volume variability meet certain criteria.

In some embodiments, the techniques described herein may be used to determine whether a subject has or is likely to have Breath Pattern Disorder (BPD). BPD, also known as Dysfunctional Breathing, is a condition characterized by irregular breathing patterns that deviate from the normal, efficient rhythm, which in many cases cannot be attributed to a specific medical cause, and lead to various symptoms. Measuring breathing in a lab setting is often an inadequate diagnostic method as BPD manifests itself when a subject is not cognizant of, or focused on, their breath pattern. Thus, a subject in a lab setting may subconsciously alter their breathing when breathing data is collected, whereas when breathing data is collected outside the lab BPD may be evident in the recorded breathing data. Therefore, by using self-contained masks or other breath measuring apparatuses to measure breathing data continuously, over extended periods (e.g., during sleep, exercise, or otherwise during everyday life), BPD may be identified and analyzed. In some embodiments, the techniques described herein may be used to determine whether a subject has or is likely to have BPD through analysis of breathing data. For example, breathing data may be collected from a subject and analyzed to determine whether a patient has BPD. It may be determined that a patient has or is likely to have BPD based on one or more respiratory features determined from the breathing data, as described herein (e.g., when one or more respiratory features exceed a threshold). In some embodiments, it may be determined that a subject is experiencing BPD based on a frequency and/or harmonic analysis of breathing data. For example, such an analysis may indicate that the subject has no defined “peaks” in frequency of breath rate and/or other respiratory metrics, and therefore has highly irregular breathing and may be experiencing BPD.

In some embodiments, the techniques described herein may be used to determine whether a subject has or is likely to have Cheyne-Stokes respiration (CSR). CSR is an abnormal breathing pattern characterized by a cyclical pattern of breathing, alternating between periods of hyperventilation and hypopnea (underventilation), with the full cycle typically lasting 1-2 minutes. This pattern, typically occurring during sleep, is associated with heart failure as well as brain related conditions like stroke and brain tumors. Subjects at risk for CSR may wear a device that may collect breathing data (e.g., a mask equipped with a differential pressure sensor or other suitable device) during sleep, during which their breathing data may be analyzed. Using self-contained masks or other breath measuring apparatuses to measure breathing data during sleep is easier than collecting such data in a clinical environment, and allows for more data to be collected continuously, over extended periods than in a clinical environment. One or more respiratory metrics may be identified from the breathing data and may be analyzed to identify whether the subject is exhibiting breathing patterns associated with CSR. It may be determined that a subject has or is likely to have CSR when they have periods of high breath rate variability, alternating between high and low breath rates. In some embodiments, a subject may be alerted (e.g., by a device connected to the device collecting breathing data) when they are exhibiting CSR to allow them to return to a controlled breathing pattern and avoid onset of other symptoms. In some embodiments, it may be determined that a subject has or is likely to have CSR based on a harmonic analysis of breathing data. For example, a Fourier analysis of breathing data may be generated, and a peak indicative of alternating cycles of high and low breathing rates may be present at a frequency corresponding to about 100 seconds, a common period for CSR cycles. Such a peak may be used to identify that the subject is likely to be experiencing CSR.

In some embodiments, the techniques described herein may be used to track athletic performance of a subject. For example, a subject may wear a device for collecting breathing data (e.g., a mask equipped with a differential pressure sensor or other suitable device) during a workout or training protocol, during which one or more respiratory metrics and/or respiratory features may be determined, as described herein. The respiratory metrics and/or respiratory features may be displayed to the subject and/or stored for later review. The subject may review the metrics and/or respiratory features and use the indications to adapt their training. Using self-contained masks and/or breathing apparatuses to collect breathing data during exercise allows for breathing data to be effectively collected over extended periods of exercise. Such self-contained masks do not require the subject to be tethered to equipment and allow them to move about freely during exercise. Furthermore, these masks do not require exercises to be performed in a laboratory setting and can allow for data to be collected in “real world environments” (e.g., at a gym, pool, track, outdoors, among other environments).

In some embodiments, the techniques described herein may be used in guiding the treatment of one or more respiratory conditions. For example, the techniques described herein may be used in guiding breathing exercises for a subject experiencing or at risk of a particular condition. The breathing of a subject may be monitored (e.g., using a mask equipped with a differential pressure sensor or other suitable device) and respiratory metrics and/or features may be determined from breathing data recorded from the subject. The metrics and/or features may be displayed to the subject (e.g., on a user interface of a device connected to the breath measuring device). The metrics and/or features determined from the breathing of the subject may be used to determine whether the subject is performing the exercises correctly or otherwise has modified their breathing behavior in such a way as to reduce the likelihood that they will exhibit the condition going forward. One or more breath adjustments may be determined for the subject based on the respiratory metrics and/or features and displayed to the subject (e.g., “Longer Inhale” may be displayed to a subject who has increased breath rate variability due to shortened inhalations). Such guided breathing exercises may be used as part of a treatment for respiratory conditions. For example, behavioral breath therapy is frequently used in treating BPD to help regularize breathing, and the techniques described herein may be used to provide real time statistics during such therapy.

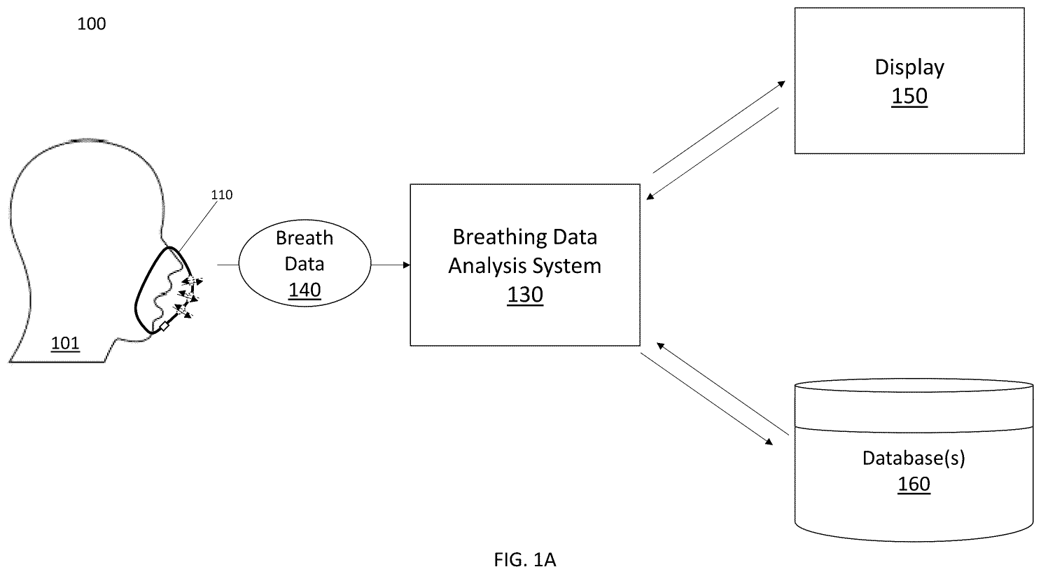

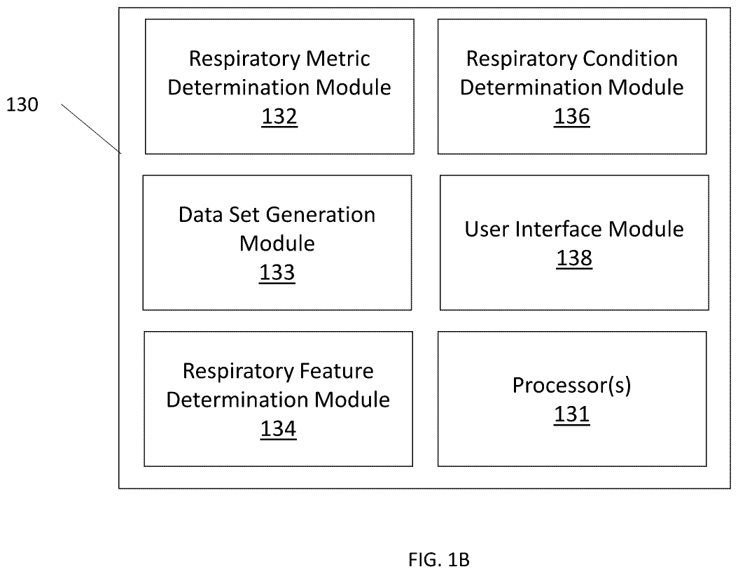

In some embodiments, the techniques described herein may be implemented as a system or in conjunction with a system, such as representative system 100 of FIG. 1A. System 100 includes mask 110, breathing data analysis system 130, user interface 132 and database(s) 134. While the components of system 100 are shown as separated, it is to be appreciated that system 100 may be implemented as a single device or as multiple interconnected devices.

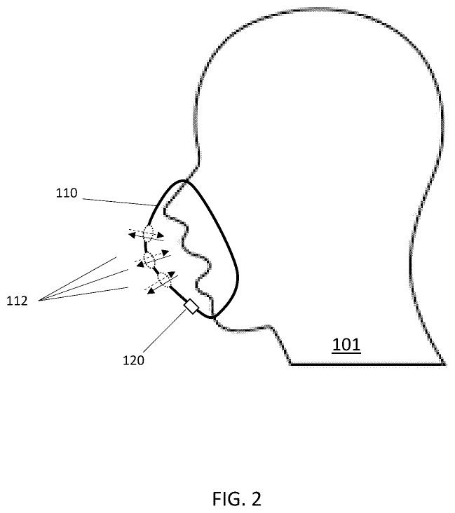

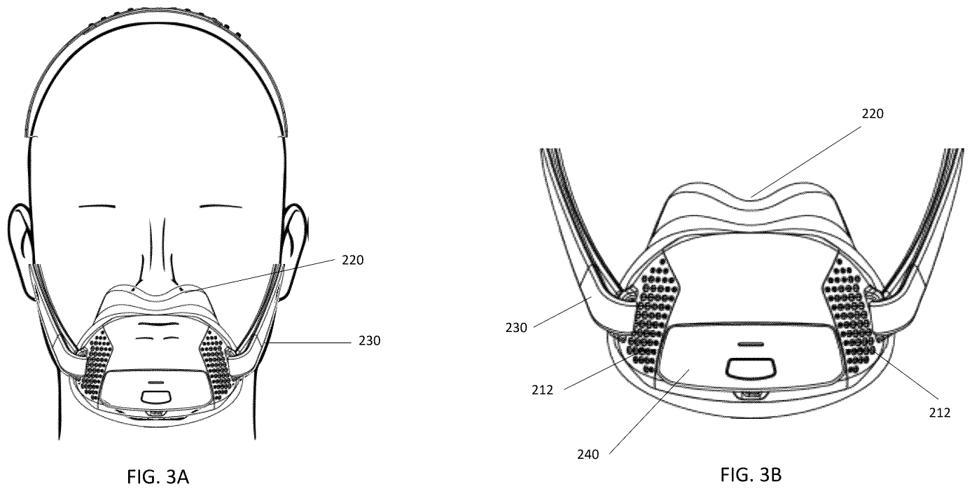

The mask 110 may be configured to collect breathing data of subject 101 and transmit the breathing data to breathing data analysis system 130. Examples of masks that may be used for capturing breathing data of a subject are discussed below with reference to FIGS. 2-3B.

The mask 110 may be communicatively coupled to the breathing data analysis system 130, such that breath data 140 may be transmitted to the breathing data analysis system. For example, the mask 110 may be wirelessly connected to breathing data analysis system 130, or alternatively the mask 110 may be connected to breathing data analysis system 130 via a wired connection (e.g., in an embodiment where the breathing data analysis system is integrated into the mask).

The breathing data analysis system 130 may comprise any suitable device(s) and/or system(s) for analyzing breathing data received from the mask 110. For example, the breathing data analysis system 130 may comprise one or more of a desktop computer, a laptop computer, a smartphone, a server, a tablet, a wearable device (e.g., a smart watch, smart glasses, smart goggles, VR headset, etc.) and/or among other devices.

The breathing data analysis system 130 is connected to display 150. The display 150 may be separate from the breathing data analysis system, such as a connected display (e.g., a television, a monitor, a device with an integrated screen, or other suitable display), or may be integrated with the breathing data analysis system as a single device. The display may allow a user of the system to view information generated by the user interface module 138 of the breathing data analysis system 130, and may additionally accept inputs from the user to control the system 100. In some embodiments, display 150 may be a stationary display that is part of an exercise related apparatus such as a treadmill, a stationary bicycle or a similar type of exercise system. In some embodiments display 150 may be a stationary display that is located close to a patient that is being monitored. In some embodiments display 150 may be separated from the mask 110 and/or breathing data analysis system, allowing displayed data to be viewed by a person who is not the subject, for example a trainer, a coach, a therapist, a medical professional or a caregiver.

The breathing data analysis system is additionally connected to database(s) 160. The databases 160 may reside on physical data storage directly connected to and/or integrated with the breathing data analysis system. In some embodiments, the databases 160 may reside on storage external to the breathing data analysis system 130 and may be accessible through a network connection, for example, the databases 160 may reside on cloud storage accessible through the internet. The databases 160 may store breathing data, respiratory metrics, respiratory features, respiratory conditions, and/or breathing recommendations, as described herein, which may be provided via the breathing data analysis system. In some embodiments, breathing data 140 may be provided to the databases 160 for storage directly from mask 110. The data stored in databases 160 may include historic data recorded from a subject and/or determined from a subject, and may be accessible to users of the system 100 for later analysis. In some embodiments, the databases may store one or more modules and/or models used for analyzing, processing and/or displaying data related to subject respiration. The data stored in databases 160 may be provided to users of system 100 for review, for example a subject may review their own data, or a clinician may review data of one or more patients to analyze respiratory conditions. The data for a subject (e.g., breathing data, respiratory metrics, respiratory features, and/or respiratory conditions) may be analyzed and used to inform the analyses, recommendations and/or diagnoses of other subjects. For example, data for a subject diagnosed with a particular condition may be reviewed and/or compared to that of a second subject, suspected of having the same condition to inform the diagnosis of the second subject.

FIG. 1B is a diagram of example components of a representative breathing data analysis system 130, according to some embodiments. The breathing data analysis system 130 may include one or more computer hardware processors 131 for analyzing breathing data 140 captured from the mask 110. The processors 131 may utilize one or more modules for analyzing breathing data 140 received from the mask 110.

The breathing data analysis system 130 may determine one or more metrics from the breathing data 140 received from the mask 110, for example using respiratory metric determination module 132. The respiratory metric determination module 132 may be configured to process the breathing data received from the mask to determine one or more of a: breath rate, breath duration, inhaled volume, exhaled volume, peak inhalation flow rate, peak exhalation flow rate and/or I/E ratio of the subject 101, among other respiratory metrics described herein. Respiratory metrics determined using the breathing data analysis system may be stored for future use and/or analysis, such as in memory of the system and/or one or more databases connected to the system, such as database(s) 160 of FIG. 1A.

The breathing data analysis system 130 includes data set generation module 133 which may be used to generate a dataset of breathing data and/or respiratory metrics determined from breathing data. The dataset may be maintained in memory of the breathing data analysis system and may be continuously updated as additional breathing data is received. The data set may be analyzed to determine one or more respiratory features, as described herein.

The breathing data analysis system 130 additionally includes respiratory feature determination module 134. The respiratory feature determination module 134 may determine one or more respiratory features of the subject from the respiratory metrics and/or breathing data. Such features may include features such as a mean, median, variance, standard deviation, percentiles, maximum value, minimum value, skewness, kurtosis, and/or variability of a respiratory metric over a period and/or between periods (e.g., time period or number of breath cycles). The respiratory features may additionally include frequency domain features determined from the breathing data and/or respiratory metrics. In some embodiments, the respiratory feature determination module may compare the respiratory metrics determined from the breathing data at different periods (e.g., time periods, different breath cycles, different phases of a breath cycle, etc.) and may determine one or more features such as variabilities for respiratory metrics based on the comparison. For example, the respiratory feature determination module 134 may determine one or more of: a breath rate variability, a breath duration variability, an inhaled volume variability, an exhaled volume variability, a peak inhalation flow rate variability, a peak exhalation flow rate variability and/or an I/E ratio variability, among other variabilities that may be determined based on respiratory metrics. Respiratory features determined using the breathing data analysis system may be stored for future use and/or analysis, such as in memory of the system and/or one or more databases connected to the system, such as database(s) 160 of FIG. 1A.

The breathing data analysis system 130 additionally includes respiratory condition determination module 136. The respiratory condition determination module may analyze one or more of: breathing data, respiratory metrics, and/or respiratory features to determine whether a subject is exhibiting one or more respiratory conditions. Such respiratory conditions may include one or more of: Obstructive Sleep Apnea (OSA), Breath Pattern Disorder (BPD), and/or Cheyne-Stokes respiration (CSR), as described herein. In some embodiments, non-medical conditions may be identified and/or analyzed based on respiratory metrics and/or features, such as exercise and/or stress. The respiratory condition determination module may output a representation of one or more respiratory conditions the subject is experiencing, likely to be experiencing and/or may experience in the future. For example, an indication of one or more conditions, a degree to which the subject may be experiencing one or more conditions, and/or a degree to which the subject may be experiencing symptoms of one or more conditions.

The breathing data analysis system 130 additionally includes user interface module 138. The user interface module 138 may be used to generate one or more user interface elements for display to a user of the system (e.g., a subject, a clinician, etc.). The user interface module 138 may generate displays of one or more of: breathing data, respiratory metrics, respiratory features, respiratory conditions, breathing recommendations and/or other information, as described herein. The user interface module 138 may additionally accept inputs from a user of the system, for example to control the mask 110 (e.g., to start/stop data collection, calibrate the mask, etc.), view breathing data, view respiratory metrics, view respiratory features, view respiratory conditions, view historic data, start/stop a breathing exercise, among other functions.

FIG. 2 depicts in more detail a representative mask 110 embodying the features described above with reference to FIG. 1A. The breathing mask 110 configured with apertures 112 for air flow, allowing a user to breath comfortably while wearing the mask, but ensuring that while the mask is worn, substantially all of the inhaled and/or exhaled air flows exclusively through the apertures (112). The respiratory air flow (AF) changes during each breath cycle, thus it is a function of time that can be positive (corresponding to exhalation) or negative (corresponding to inhalation).

The mask 110 may be secured to the user's head by any suitable means, such as a harness or a plurality of straps or bands that surround the head or the cars (not shown).

Mask 110 additionally includes high-speed differential pressure sensing device 120 that is configured to measure the difference between the instantaneous air pressure inside the mask 110 and the ambient pressure (outside the mask). This is referred to as a differential pressure (DP) measurement. Suitable 2-port sensors that measure differential measure at very high speed are known in the art and available commercially from multiple vendors, such as Honeywell, Merit Sensor, Bosch, STMicroelecrtonics, Sensirion and others. As shown, the mask 110 includes a single sensing device 120, but may include multiple sensing devices.

The mask 110 may be designed such that there is a reliable, consistent relationship between DP and AF through the mask. Different characteristics of the mask 110 may be selected to control this relationship, for example the shape of the mask 110, locations of the apertures 112, and the location of the DP sensor 120 within the mask may be selected to control the relationship between DP and AF through the mask. The relationship between DP and AF may be monotonic so that for a particular design of mask and apertures, a greater air flow rate corresponds to greater differential pressure. It is also generally the case that reversing the air flow direction between exhalation and inhalation reverses the sign of DP (between positive and negative).

The mask 110 may include an electronic circuit for receiving breathing data of the subject which may include the DP signals generated by the DP sensor 120. In some embodiments this circuit is attached to the mask. The breathing data received by the circuit may be stored, processed and/or transmitted to other data processing systems, such as breathing data analysis system 130 of FIG. 1A. The breathing data may be transmitted via wireless connection to a receiving system, allowing untethered mobility for the person wearing the mask. Any suitable wireless communications protocol may be utilized, whether now known or later developed, including but not limited to Bluetooth®, BLE, WiFi, Zigbee, NFC and LoRa®. The breathing data may alternatively be transmitted through a wired connection. The circuit and the sensors are powered by a power supply or a battery; in certain embodiments a small rechargeable or replaceable battery is preferred so as to enable an untethered and lightweight system which may be worn by the subject over extended periods. The power supply or battery may be rechargeable and may have sufficient battery life to power the electronics over extended periods (e.g., 1, hour, 5 hours, 10 hours, 15 hours, 20 hours, or greater than 20 hours).

The DP sensor 120 is configured to capture pressure readings at a sufficiently high rate, to provide the required temporal detail of AF. Commercially available piezoelectric DP sensors can collect hundreds of readings per second or more. Given the physiology and kinetics of human breath, that level of temporal resolution may exceed the requirements for analyzing AF in subjects. A more typical requirement for AF analysis may be between 10-100 readings per second, which is well within the capabilities of many existing piezoelectric DP sensors.

FIGS. 3A-B depict a more detailed representation of a representative mask that can be worn over extended periods and in daily life to collect breathing data of a subject, according to some aspects of the technology described herein. FIG. 3A depicts the mask 220 attached to a subject's head though straps 230 that wrap around the subject's head, and FIG. 2B depicts the mask 220 detached from the subject's head. Mask 220 includes an array of small apertures 212 that allow for air flow into and out of the mask, such as described above with reference to mask 110. Further, as shown the mask 220 mates with the bottom of the nose but does not cover the bridge of the nose. The mask 220 additionally includes electronics 240 that may include one or more differential pressure sensors and circuitry for receiving, storing and/or transmitting breathing data recorded by the differential pressure sensors, such as described above with reference to mask 110. The mask 220 may additionally include a battery for powering the electronics 240. The battery may be rechargeable and may have sufficient battery life to power the electronics over extended periods (e.g., 1, hour, 5 hours, 10 hours, 15 hours, 20 hours, greater than 20 hours).

The mask 220 may additionally include ergonomic features to allow the mask to be comfortably worn over extended periods. For example, the mask 220 may be lightweight (e.g., approximately 100 g, approximately 75 g, approximately 50 g, approximately 30 g, or less than 30 g in weight). Such a low weight may be achieved through a small footprint and the components of the mask. The mask 220 has a small footprint on the face of the subject, and does not cover the bridge of the nose. Further, the mask 220 may include a physically small battery and use very low power sensors and digital electronics to achieve extended data collection and wireless operation. These features, among others, make the system more conducive to extended time measurements, which result in more extensive breath data. In some embodiments the collection of more extended data is important for the statistical significance of variability analysis, which is explained below in greater detail.

In some embodiments, data which is collected over periods of time may be using masks 110 or 220 may provide respiratory information like breath count, rate and volume, as well as oxygen consumption and carbon dioxide (CO₂) production. The components of the masks may further include additional sensors including gas sensors, temperature sensors, chemical sensors, electromechanical sensors, optical sensors, accelerometers, and/or humidity sensors.

The masks 110 and 220, described with reference to FIGS. 2 and 3A-B, respectively, are shown by way of example only and the techniques described herein may be used in conjunction with any suitable apparatus for measuring the breathing of a subject.

In some embodiments the system may be calibrated before it is used to improve the accuracy of data collected from a subject. Calibration is not required to collect breathing data from a subject, and a system may be characterized such that a relationship between air flow and differential pressure recordings are known. Calibration of the system may be performed using a calibration fixture that is configured to provide a known air flow rate, while measuring the DP reading of the sensor 120 on mask 110. In some embodiments the fixture may be shaped like a human head (a “mannequin”), with air flowing through a modeled mouth and/or nose, through a conduit or tube that is attached to a source of air flow. Examples of a source of air flow include, but are not limited to, a pressurized air tank, a continuous air pump, and/or a mechanical bellows. The fixture used for calibration need not have the full anatomical structure of a human head or face, but only an air conduit with a structure or topography that may mate to the mask 110 forming an approximate seal so as to direct air to flow exclusively through the mask's apertures. During calibration, the air flow may be further controlled by a manual or electric regulator or mass flow controller. A flow sensor measuring the air flow may be configured at any point along the airflow path into the mask. The flow may be measured by an in-line flow meter. Such in-line flow meters are commercially available in various performance ranges.

When calibrated, the DP sensor 120 may provide a substantially instantaneous indication of the flow rate through the mask apertures, which is substantially equal to the co-instantaneous breath flow rate of a subject wearing the mask 110. The DP readings may be collected along with their precise timing and are then available for further analysis and communication. The DP readings and their timings may be transmitted to breathing data analysis system 130 as breath data 140, as described herein.

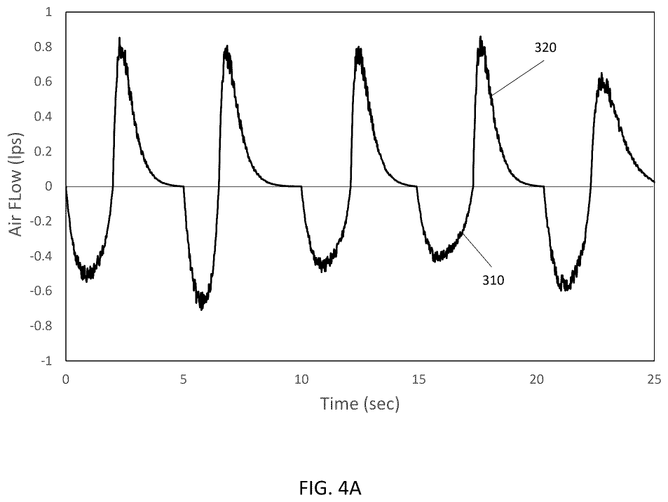

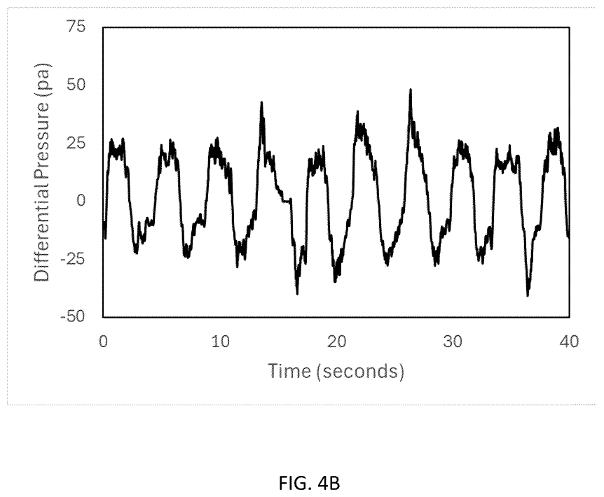

FIG. 4A shows an example of AF readings of a subject over the course of five breaths, collected using a system such as system 100 described herein. The chart depicts in high resolution air flow (vertical axis) vs time (horizontal axis, in seconds). The regions with positive air flow values 320 correspond to exhalation while negative values 310, namely reversed direction, correspond to inhalation. The shape of the inhalation curves is different from the exhalation curves, although the cumulative volume of air is generally similar. The detailed profile of flow rate over the course of multiple breaths, such as shown in this figure, presents a rich set of data that can be used for various diagnostic and research purposes. For example, respiratory metrics may be determined from the data, such as a breath rate, breath duration, inhaled volume, exhaled volume, peak inhalation flow rate, peak exhalation flow rate and/or I/E ratio. These metrics may be further analyzed, as described herein, to determine respiratory features and/or conditions a subject may be exhibiting.

FIG. 4B shows a different example of breathing data collected from a subject. The data of FIG. 4B includes DP readings collected at 50 data points per second from a subject using a system, such as system 100 described herein, while riding a stationary bicycle. The data represents a 40 second time segment with 10 breaths shown. Such data may be analyzed, as described herein, such as to measure the athletic performance of the subject. The subject may compare their breathing data shown in FIG. 4B to past or future breathing data collected during the same exercise to evaluate their progress.

The breathing data, such as shown in FIGS. 4A-B may be used in determining one or more respiratory metrics for a subject. The breathing data may be used in determining breath duration (i.e. seconds per breath) and corresponding rate or frequency (breaths per minute), for example by identifying the breathing cycles (inhalation and exhalation) present in a sample of breathing data. Breath frequency is a strong indicator of physical and mental stress or stimulation and can be used to monitor a person's level of stress or exertion. Increases in breathing rate and decreases in breath duration are associated with increased stress and exertion in individuals. The quantitative changes induced by physical stress, as well as mental or emotional stress, to the breath rate vary significantly among individuals and can be modified through conditioning or therapy. For example, when a subject is experiencing high stress or exertion, he/she may be guided through breathing exercises to return to a lower-stress state.

In some embodiments, respiratory air volume—both volume per breath and volume per minute—may be determined from DP measurements obtained from a subject. As described above, a mask having a differential pressure sensor may be used to record breathing data. The relationship between air flow into and out of the mask may be characterized, such as based on the design of the mask and/or through a calibration procedure. This relationship may be used to determine air volume measurements from differential pressure measurements recorded by a mask. The volume per breath may be determined by identifying breath cycles in the differential pressure data and determining, based on the change in differential pressure over the breath cycle the volume inhaled and/or exhaled by the subject. The air volume over a time period may be used by determining the total volume inhaled and/or exhaled over a particular time period of recorded DP data (e.g., 10 second, 30 second, 1 minute, 2 minutes, 5 minutes, etc.) Air volume per minute, also known as “minute ventilation” and usually denoted as VE, is an important physiological indicator for estimating energy use and assessing respiratory health or cardiovascular conditioning.

Additional metrics may be determined from the breathing data recorded by a mask or other apparatus for measuring breathing. In some embodiments the rate of increase of air flow at the beginning of each breath cycle and/or the rate of decrease of air flow at the end of breath cycles may be determined from breathing data recorded from a subject. In some embodiments the peak value of the flow rate during each breath cycle may be determined from breathing data recorded from a subject. In some embodiments the time from start of inhalation and/or to the peak flow during that phase of the breathing cycle may be determined from breathing data recorded from a subject. In some embodiments the ratio between the duration of inhalation and the duration of exhalation (the I/E ratio) in the same breath may be determined from breathing data recorded from a subject.

In some embodiments, respiratory metrics may be determined over extended periods, such as time periods or multiple breath cycles. In some embodiments the notation Xᵢ is used to describe the value of a certain respiratory metric, where for each value of the index i (=1, 2, 3, . . . ), Xᵢ describes a particular metric. For example, X₁ may represent breath duration, X₂ may represent the I/E ratio, etc. The plurality of variables Xᵢ=X₁, X₂, X₃, X₄, X₅, etc. may represent, in no particular order, any of a group of respiratory metrics that includes, but is not limited to, breath rate, breath duration, inhaled volume, exhaled volume, peak inhalation flow rate, peak exhalation flow rate and I/E ratio. Furthermore, the notation may take the form X₁(n) to include a breath counter n, so that it represents the value of that metric Xi during the n^{th} breath. For example, if X₃ is chosen to represent the exhaled volume, then X₃(12) is the exhaled volume on the 12^{th} breath, etc.

In some embodiments, one or more respiratory metrics X; (n) may be analyzed over multiple breaths to extract respiratory features that may be used in analyzing the respiratory condition of a subject and/or determining one or more conditions exhibited by the subject. In some embodiments the mean, or average, value may be determined for one or more respiratory metrics, which may be calculated over a certain duration of time and/or over a series of consecutive breaths. As non-limiting illustrative examples, these can be the mean of the peak inhalation air flow over the course of 10 breaths, or the mean of the I/E ratio over an extended time period. As described herein, the systems including minimally invasive masks such as 110 or 220 may be worn for extended periods of time to allow for large amounts of breathing data to be collected from subjects and thus for statistics to be determined based on the collected breathing data.

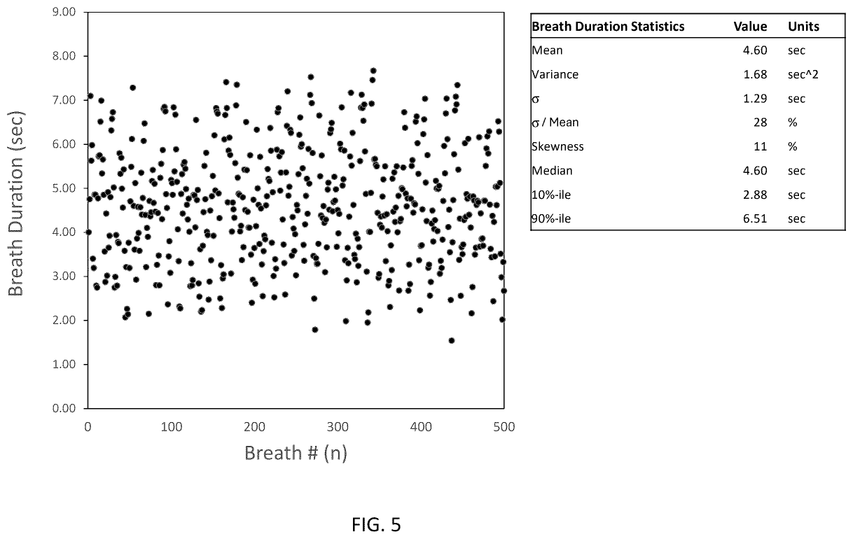

FIG. 5 shows a chart depicting a representative sequence of 500 breaths, each breath represented as a point on the chart with the y-axis representing the duration of that breath in seconds. Next to the chart is a table summarizing a subset of the features associated with the duration of these breaths, including the mean, the variance, the standard deviation, as well as the 10^{th} and 90^{th} percentile. The average breath rate, defined in terms of breaths per minute (BPM), is 60 (seconds) divided by the mean breath duration in seconds. For example, if each breath takes 10 seconds, the breath rate is 60/10=6 BPM.

In some embodiments, respiratory features may include maximum, minimum and median value, and more generally, percentile values, which may be used to represent the distribution range of any of these metrics. As a nonlimiting illustrative example, the 80^{th} percentile of the breath duration corresponds to a value (in seconds) for which 80% of recorded breaths are shorter in duration, and 20% are equal to or longer in duration.

In some embodiments, respiratory features may include higher statistical moments which may be calculated and used to characterize the distribution of respiratory metrics. For example, the mean value mentioned earlier is in fact the first-order moment. Each Xᵢ has a mean value which is of interest. The 2^{nd} order moments are the variance Var(Xᵢ) and, by extension, its square root which is the Standard Deviation or S(Xᵢ). Higher statistical moments include skewness (a 3^{rd} moment) that characterizes asymmetry around the mean, and kurtosis (a 4^{th} moment), a measure of deviation from normal (Gaussian) distribution. Higher moments may also be determined from breathing data recorded from a subject. The ability of the systems described herein to apply these statistical methods to respiratory data may depend on acquiring reliable, high resolution breath data over extended periods of time. As described herein the use of minimally invasive masks such as 110 or 220 allow for collection of breathing data over extended periods of time and outside of clinical settings that may be used for characterizing breathing of subjects and identifying respiratory conditions the subjects may exhibit. These large amounts data for analysis that were previously unavailable using more invasive measurement apparatuses, as such apparatuses cannot be used to effectively capture data from subjects during everyday life, where a subject's breathing is least impacted. However, it is to be appreciated that the techniques described herein are not limited to data collected using a particular breathing measurement apparatus.

Additional analyses may be performed on respiratory metrics determined from breathing data to determine one or more respiratory features for the subject. A non-limiting example is a normalized breath rate variability (BRV) feature, which may be determined from breath data. In some embodiments, BRV may be the standard deviation (STDEV) of a series of recorded breath durations. In some embodiments BRV may be represented as the ratio of the STDEV to the mean breath duration.

In some embodiments the breath rate variability may be expressed in terms of a score Z. For example, Z may be computed from a series of breaths and defined in such a manner so that its value always falls between zero and 1 (or 100%), such that if the breaths are perfectly uniform, Z=100%. This score may be referred to “breath rate uniformity”. A higher Z may be indicative of more uniform breath, whereas a lower Z suggests a more erratic breathing pattern, which may be indicative of an underlying physical or emotional condition.

In some embodiments breath rate variability may be determined by determining the distribution of the difference between pairs of successive breaths (“successive difference”), which is generally known in the art as Poincaré analysis.

Similarly to BRV, breath volume variability and peak flow variability features may be tracked as indicators of stable breathing patterns. These variability features, and variability features for any other respiratory metric may be determined as described herein, such as with reference to the BRV of a subject.

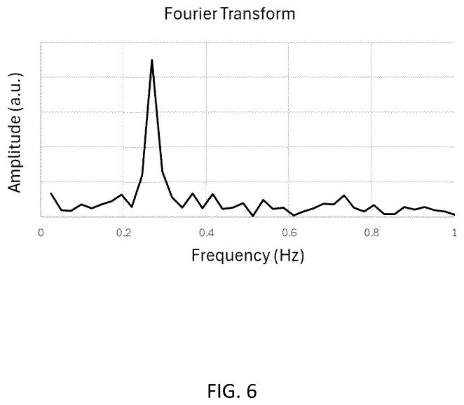

In some embodiments, respiratory features determined for a subject may include frequency analysis of breathing data may be, which may be indicative of breathing regularity and used in identifying respiratory conditions present in subjects. As breathing is generally a periodic phenomenon, harmonic and/or Fourier analysis may be used to determine one or more respiratory features from breathing data. Fourier analysis may be used on time-resolved air flow data (AF), as opposed to breath-by-breath data like breath volume and duration. The higher the rate of the flow measurement, the higher the quality may be of the Fourier analysis. In some embodiments, the measurement is repeated 10 times per second (10 Hz). In some embodiments, the measurement is repeated at a rate that is between 10-50 Hz. In some embodiments the measurement is repeated at a rate that is higher than 50 Hz. FIG. 6 shows an example of a Fourier transform of the same breath data shown in FIG. 4B, which is collected with a rate of 50 Hz. The distinct narrow peak at 0.25 Hz clearly represents the relatively uniform breath rate of 4 breaths per second, as seen in FIG. 4B; less uniform breath would manifest itself as a wider peak. As described herein, irregularities and non-uniformities in breathing may be indicative of one or more respiratory conditions.

In some embodiments, respiratory features determined for a subject may include a harmonic analysis of breathing data. For example, air flow data may be analyzed via a harmonic analysis, and a harmonic analysis of the AF(t) results in a harmonic representation

$$A F ( t ) = \sum {A}_{n} sin ( 2 π f \times n \times t )$$

The harmonic coefficients {Aₙ} may represent the breathing pattern of the subject in terms of frequency components (e.g., f, 2f, 3f, etc.). In some embodiments, a Fourier transformation may be applied to AF data, which treats the frequency as a continuum, regardless of an approximate periodicity in the AF data. Airflow may be represented by its Fourier transform function, typically using the angular frequency ω=2πf, giving

$$A F ( ω ) = \int A F ( t ) {e}^{- i ω t} dt .$$

In some embodiments a Fourier transform may be used in determining whether a subject has or is likely to have one or more respiratory conditions and/or in tracking the progression of one or more respiratory conditions. In regular breathing there is expected to be a distinct peak in the Fourier transform AF (@) around a frequency corresponding to the breath rate (RR), namely ω≈2πRR. The more uniform a subject's breath is, the sharper the Fourier peak will be at the corresponding frequency. In some embodiments, symptoms of Cheyne-Stokes respiration (CSR) may be identified using a Fourier analysis of respiratory data. For example, a subject having or likely to have CSR may show a subharmonic peak in the Fourier transform AF(ω) of their breathing data, broadly around a value of approximately 0.01 Hz, which would correspond to a CSR cycle (alternating between periods of high and low breath rates) of about 100 seconds, which is a common period for such symptoms of CSR. In some embodiments, symptoms of Breath Pattern Disorder (BPD) may be identified using a Fourier analysis of respiratory data. For example, for a subject experiencing, or likely to be experiencing BPD, the Fourier peak may be barely present or entirely non-existent due to very wide variability of the respiratory rate in the subject's breathing.

The respiratory features described above, including a mean, median, variance, standard deviation, percentiles, maximum value, minimum value, skewness, kurtosis, and/or variability of a respiratory metric over a period and/or between periods (e.g., time period or number of breath cycles) and/or frequency domain features, may be used in determining and/or monitoring the progression of one or more respiratory conditions of a subject. As noted above, such conditions may include one or more of: Obstructive Sleep Apnea (OSA), Breath Pattern Disorder (BPD), Cheyne-Stokes respiration (CSR), and/or non-medical conditions such as exercise, respiratory exertion and/or stress. By using non-invasive apparatuses to collect breathing data of subjects over extended periods, these conditions can be better understood and identified in individuals. The features described herein, including variabilities of respiratory metrics previously were not analyzed over extended periods and therefore could not be used in identifying or monitoring such conditions.

Particular conditions may be determined when one or more respiratory features for a subject and/or a combination of respiratory features for a subject satisfy one or more criteria associated with the condition. For example, it may be determined a subject has or is likely to have OSA when a breath rate variability, an inhaled volume variability, and/or an exhaled volume variability exceeds respective thresholds, as OSA is characterized by irregularities in breath rate and volume during sleep. It may be determined a subject has or is likely to have BPD when one or more respiratory metric variabilities exceed respective thresholds, as BPD is characterized by general irregularities in breath cycles. It may be determined a subject has or is likely to have CSR when they have periods of high breath rate variability, alternating between high and low breath rates, as CSR is characterized by extreme swings in breath rate. It may be determined a subject is exercising based on an increase in breath rate and/or volume, and it may be determined that a subject is reaching respiratory fatigue when one or more respiratory metric variabilities satisfy certain criteria, as this indicative of a subject no longer being capable of maintaining a regular breathing pattern.

In some embodiments, the criteria for particular respiratory features used in determining whether a subject has or is likely to have a particular condition are predetermined. In some embodiments, the criteria for particular respiratory features are unique to a subject and may be determined based on baseline measurements collected from a subject. In some embodiments, the criteria for particular respiratory features used in determining whether a subject has or is likely to have a particular condition are derived, such as based previously recorded breathing data concerning the subject and/or other individuals.

In some embodiments, the respiratory metrics and/or features determined from breathing data may be used to monitor additional medical conditions, including but not limited to pulmonary maladies such as congestive obstructive pulmonary disease (COPD), obstructive apnea, pulmonary fibrosis, tumors, pneumonia, pulmonary inflammation, and edema. In some embodiments the respiratory metrics and/or features determined from breathing data may be used to monitor medical conditions that are not directly associated with the lungs, including but not limited to neurological disorders, stress, infectious diseases, and any psychological/psychiatric, metabolic, muscular, immunological, nutritional and endocrinological conditions.

In some embodiments, respiratory metrics and/or features may be represented as indications a subject has, is likely to have, or may exhibit a particular condition in the future. For example, if it is determined based on an analysis of breathing data that a subject is likely to be experiencing CSR, an indication may be provided to the subject (e.g., on a display) that they are likely experiencing CSR. In some embodiments, respiratory metrics and/or features may be represented as a degree to which a subject is experiencing a particular condition and/or symptoms of a particular condition. For example, a score indicative of the regularity of one or more breathing metrics may be output to a subject experiencing BPD, such that they may understand how their symptoms are progressing.

In some embodiments the respiratory metrics, features, and/or conditions determined from breathing data may be reported in a non-medical context, including (but not limited to) tracking mental and physical wellness and conditioning, exercise, sleep, and biofeedback.

In some embodiments data, including breathing data, metrics, features, and/or conditions determined from breathing data may be presented to a user on a display (e.g., display 150 of FIG. 1A). In some embodiments, data may be made available remotely for review, allowing data to be viewed by a person who is not the subject, for example a trainer, a coach, a therapist, a medical professional or a caregiver.

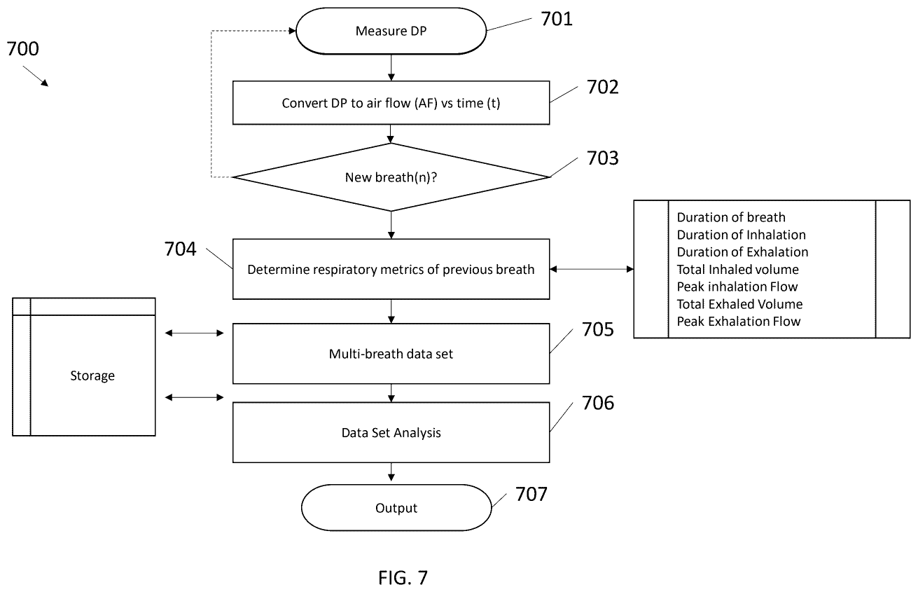

FIG. 7 is a flow chart of a representative process that may be performed by a system, such as by system 100 of FIG. 1A, to determine respiratory metrics, features and/or conditions of a subject.

Process 700 begins with act 701, at which differential pressure is measured. This may be performed in any of numerous ways. In some embodiments, differential pressure may be measured using one or more differential pressure sensors, such as those on masks 110 and 220, described herein. The masks may be affixed to patients and may be worn over continuous periods of time. The masks may be worn during sleep, exercise, or otherwise during everyday life to record differential pressure from a subject. The differential pressures recorded from the subject may be indicative of the respiration of the subject.

At act 702, the differential pressure data is converted to air flow vs time. This conversion may be performed using any suitable technique, such as is described above, and may be performed using processing circuitry of a mask and/or by a breathing data analysis system as described herein. In some embodiments, the conversion may be made based on a calibration of the mask to determine the relationship between differential pressure recorded by the mask and a known airflow, as described herein. In some embodiments, the relationship between differential pressure and airflow through a mask may be characterized by the design of the mask, as described herein, and may be used in converting the differential pressure data to air flow data.

At act 703, a determination is made whether there is a new breath occurring. This may be performed in any of numerous ways, such as based on signals from the differential pressure sensors, which may detect the breathing of the subject as changes in differential pressure. If the subject is no longer using the mask, the differential pressure will not change, and so it may be determined that there is not a new breath. Act 703 may continuously be performed as a subject is wearing a mask, such that breathing data is continuously collected for the subject. The remaining acts of process 700 may be performed using previously collected breathing data and as additional data is collected. In some embodiments, act 703 may not be performed and differential pressure data may be continuously recorded using a mask. In some embodiments, a mask may begin recording differential pressure data when the mask is worn by a user (e.g., when a user manually turns the mask on or selects an input to begin recording, and/or when it is detected the mask has been donned by a user such as through one or more sensors).

At act 704 one or more respiratory metrics are determined for a previously recorded breath, using the breathing data for the subject, represented as air flow over time. The respiratory metrics may be determined using a respiratory metric determination module, such as module 132 of FIG. 1B. FIG. 7 includes examples of respiratory metrics that may be determined at act 704, including a duration of breath, a duration of inhalation, a duration of exhalation, a total inhaled volume, a peak inhalation flow, a total exhaled volume and a peak exhalation flow. It is to be appreciated that additional metrics may be determined, as described herein, or fewer metrics may be determined. The respiratory metrics may be determined through analysis of the breathing data received from the mask, including the air flow vs. time data obtained from the differential pressure data recorded by the mask.

At act 705, a multi-breath data set is generated from the breathing data and respiratory metrics for the subject. The multi-breath data set may be generated using a data set generation module such as 133 of FIG. 1B. The multi-breath data set may include the breathing data and/or respiratory metrics determined for a subject over time. The multi-breath data set may be continuously updated as additional data and/or respiratory metrics are received. The multi-breath data set may be organized by time and/or by breath cycle. The multi-breath data set may be stored, such as in database(s) 160 of FIG. 1A, for later use and/or analysis. The multi-breath data set may additionally or alternatively be maintained in memory of a breathing data analysis system. The multi-breath data set may have any suitable data structure, for example a vector structure or a matrix structure.

At act 706, the multi-breath data set is analyzed. The data set may be analyzed as described herein, for example to determine one or more respiratory features for a subject. The respiratory metrics for the subject may be compared over different periods (e.g., time periods and/or breath cycles). The respiratory features may include those features described herein, including a mean, median, variance, standard deviation, percentiles, maximum value, minimum value, skewness, kurtosis, and/or variability of a respiratory metric over a period and/or between periods (e.g., time period or number of breath cycles). The respiratory metrics and/or features may be further analyzed to determine whether the subject has or is likely to have one or more respiratory conditions, as described herein. Such conditions may include one or more of: Obstructive Sleep Apnea (OSA), Breath Pattern Disorder (BPD), Cheyne-Stokes respiration (CSR), congestive obstructive pulmonary disease (COPD), and/or pulmonary fibrosis. The respiratory metrics and/or features may additionally or alternatively be analyzed to determine one or more non-medical conditions of a subject such as stress, respiratory exertion and/or exercise. The respiratory metrics and/or features may additionally be analyzed to guide a breathing exercise of the subject and/or to determine whether the subject is performing the exercises correctly or otherwise has modified their breathing behavior in such a way as to reduce the likelihood that they will exhibit the condition going forward. Act 706 may be performed by a respiratory feature determination module and/or a respiratory condition determination module, such as 134 and 136 of FIG. 1B. The respiratory features and/or conditions determined for a subject may additionally be stored for later use and/or analysis.

At act 707, outputs may be provided to a subject and/or user of the system. The outputs may be provided on a user interface, as described herein and may be generated by a user interface module of the system such as 138 of FIG. 1B. The outputs may include one or more of: breathing data, respiratory metrics, respiratory features, and/or respiratory conditions of a subject. The outputs may additionally include one or more recommendations for a subject and/or guided breathing exercises for the subject.

In some embodiments, one or more machine learning models may be used in analyzing breathing data recorded from a subject, respiratory metrics and/or respiratory features determined from breathing data. The machine learning models may include one or more of: classification models, artificial neural networks, recurrent neural networks, convolutional neural networks, and/or transformer-based models (e.g., BERT, ChatGPT), among other suitable machine learning models. In some embodiments, the machine learning models may be trained to identify one or more respiratory conditions exhibited by a subject, based on breathing data, respiratory metrics and/or respiratory features determined from breathing data recorded over an extended period from one or more subjects having known respiratory conditions. The one or more ML models may be maintained in storage (e.g., databases 160 of FIG. 1A) and may be updated as additional data becomes available.

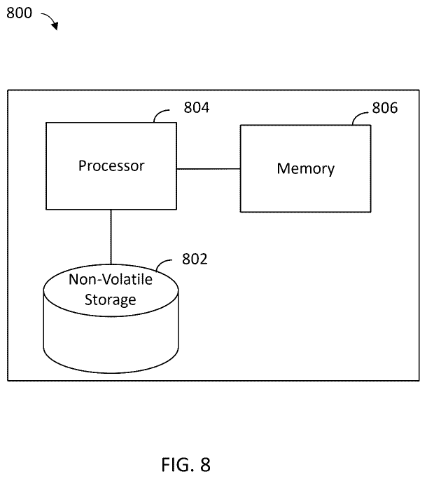

FIG. 8 illustrates a block diagram of an illustrative computing system that may be used in implementing some aspects of the technology described herein. For example, any of the computing devices described herein (e.g., 130, 150) may be implemented using computer system 800. The computer system 800 may include one or more computer hardware processors 804 and one or more articles of manufacture that comprise non-transitory computer readable storage media, for example, one or more volatile storage devices 806 (e.g., random access memory or any other suitable type of memory) and/or one or more non-volatile storage devices 802 (e.g., a hard disk, a flash memory, etc.). The hardware processor(s) 804 may control writing data to and reading data from the volatile storage device(s) 806 and the nonvolatile storage device(s) 802 in any suitable manner. To perform any of the functionality described herein, including with respect to any process described herein, the hardware processor(s) 804 may execute one or more processor-executable instructions stored in one or more non-transitory computer-readable storage media (e.g., the volatile storage device(s) 806 and/or non-volatile storage device(s) 802), which may serve as non-transitory computer readable storage media storing processor-executable instructions for execution by the hardware processor(s) 804.

The technology described herein is operational with numerous other general purpose or special purpose computing system environments or configurations. Examples of well-known computing systems, environments, and/or configurations that may be suitable for use with the technology described herein include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The technology described herein may also be practiced in distributed computing environments where tasks are performed by remote processing devices that are linked through a communications network. In a distributed computing environment, program modules may be located in both local and remote computer storage media including memory storage devices.

Having thus described several aspects of at least one embodiment of the technology described herein, it is to be appreciated that various alterations, modifications, and improvements will readily occur to those skilled in the art. Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, a tablet computer, a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store)). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion among a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIG. 7. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within +20% of a target value in some embodiments, within +10% of a target value in some embodiments, within +5% of a target value in some embodiments, within +2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A system for gathering respiratory information concerning a subject, the system comprising:
a mask, comprising a shell and a seal, the seal being arranged to contact a face of the subject and to circumscribe an area of the face when the mask is worn by the subject, the area including a mouth of the subject, the seal and the shell separating an interior space about the area of the face from an ambient space, the shell having a plurality of breathing apertures configured to allow air to flow between the ambient space and the interior space;
at least one differential pressure sensor, coupled to the mask, configured to measure a difference in air pressure between the ambient space and the interior space;
at least one computer hardware processor communicatively coupled to the at least one differential pressure sensor; and
at least one non-transitory computer readable storage medium storing instructions which, when executed by the at least one computer hardware processor, cause the at least one computer hardware processor to:
determine, based at least in part on measurements by the at least one differential pressure sensor, a plurality of metrics each concerning one or more breaths taken by the subject;
compare the plurality of metrics to determine one or more respiratory features of the subject; and
based at least in part on the one or more respiratory features, identify at least one respiratory condition which the subject exhibits or is at risk of exhibiting.

2. The system of claim 1, wherein at least one of one or more respiratory features of the subject relates to uniformity of at least one of the plurality of metrics.

3. The system of claim 1, wherein the instructions cause the at least one computer hardware processor to determine, based at least in part on measurements by the at least one differential pressure sensor, a respiratory air flow of the subject.

4. The system of claim 1, wherein a first metric of the plurality of metrics relates to one or more breaths taken by the subject during a first time period, a second metric of the plurality of metrics relates to one or more breaths taken by the subject during a second time period, and the first time period does not overlap with the second time period.

5. The system of claim 1, wherein a first metric of the plurality of metrics relates to one or more breaths taken by the subject during a first time period, a second metric of the plurality of metrics relates to one or more breaths taken by the subject during a second time period, and the first time period and the second time period are non-consecutive.

6. The system of claim 1, wherein a first metric of the plurality of metrics relates to one or more breaths taken by the subject during a first time period, a second metric of the plurality of metrics relates to one or more breaths taken by the subject during a second time period, and either or both of the first time period and the second time period comprise a predetermined amount of time.

7. The system of claim 1, wherein a first metric of the plurality of metrics relates to a first one or more breathing cycles by the subject, and a second metric of the plurality of metrics relates to a second one or more breathing cycles by the subject.

8. The system of claim 7, wherein either or both of the first one or more breathing cycles and the second one or more breathing cycles comprises a predetermined number of breathing cycles.

9. The system of claim 1, wherein at least one of the plurality of metrics relates to one or more of: breath rate, breath duration, inhaled volume, exhaled volume, peak inhalation flow rate, peak exhalation flow rate and inspiratory to expiratory ratio (I/E ratio).

10. The system of claim 1, wherein the comparing comprises:
calculating a first score representing a first one or more metrics of the plurality of metrics and a second score representing a second one or more metrics of the plurality of metrics and comparing the first score and the second score to determine the one or more respiratory features of the subject.

11. The system of claim 1, wherein a first metric of the plurality of metrics quantifies a difference between measures concerning pairs of successive breaths in a first time period, a second metric of the plurality of metrics quantifies a difference between measures concerning pairs of successive breaths in a second time period, and the comparing comprises comparing at least the first metric and the second metric to determine the one or more respiratory features of the subject.

12. The system of claim 1, wherein at least two metrics of the plurality of metrics are the same, and each concerns one or more breaths taken by the subject during a different time period.

13. The system of claim 1, wherein the comparing comprises determining variability of one or more metrics of the plurality of metrics over time.

14. The system of claim 13, wherein the variability relates to one or more of a breath rate variability, a breath duration variability, an inhaled volume variability, an exhaled volume variability, a peak inhalation flow rate variability, a peak exhalation flow rate variability and/or an I/E ratio variability.

15. The system of claim 1, wherein the at least one respiratory condition includes one or more of: Obstructive Sleep Apnea (OSA), Breath Pattern Disorder (BPD), Cheyne-Stokes respiration (CSR), congestive obstructive pulmonary disease (COPD), pulmonary fibrosis, exercise and/or stress.

16. The system of claim 1, wherein:
the comparing comprises:
comparing one or more of a breath rate, inhaled volume and exhaled volume of the subject over time to determine one or more of a breath rate variability, an inhaled volume variability, and an exhaled volume variability; and
the identifying comprises:
identifying OSA as a condition which the subject exhibits or is at risk of exhibiting when one or more of the breath rate variability, the inhaled volume variability, and/or the exhaled volume variability satisfy one or more respective criteria.

17. The system of claim 1, wherein:
the comparing comprises:
comparing one or more metrics of the plurality of metrics to determine variabilities of the one or more metrics over time; and
the identifying comprises:
identifying BPD as a condition which the subject exhibits or is at risk of exhibiting when one or more of the variabilities satisfy one or more criteria.

18. The system of claim 1, wherein:
the comparing comprises:
comparing a breath rate of the subject over time to determine a breath rate variability; and
the identifying comprises:
identifying CSR as a condition which the subject exhibits or is at risk of exhibiting when the breath rate variability satisfies one or more criteria.

19. The system of claim 1, wherein the instructions cause the at least one computer hardware processor to identify at least one treatment for the condition which the subject exhibits or is at risk of exhibiting.

20. The system of claim 1, wherein the instructions cause the at least one computer hardware processor to generate an alert responsive to identifying the at least one respiratory condition which the subject exhibits or is at risk of exhibiting.
